SEQUENCE LISTING (71 sequences)

SEQ ID NO: 1 - DNA, 63 bp, Homo sapiens
  misc_feature (32)..(32): n is a or t
gccagactcttggaaggagatgaaaaagagtncaatgctgatggaggaaagatgccaattaaa

SEQ ID NO: 2 - DNA, 63 bp, Homo sapiens
  misc_feature (32)..(32): n is c or t
tgtagagctgatgagttcctttgcaataattntctctgcaaactacatttctgggtgtgtgat

SEQ ID NO: 3 - DNA, 63 bp, Homo sapiens
  misc_feature (33)..(33): n is a or c
aaaaaccccgcagagagagcagatttgaagcanctcatggttcatgcttttatcaagagatct

SEQ ID NO: 4 - DNA, 63 bp, Homo sapiens
  misc_feature (32)..(32): n is c or t
cacccagcaatacgaatggcaccgagtcttanatttaaagaaaaagtaacaagccttaaattt

SEQ ID NO: 5 - DNA, 63 bp, Homo sapiens
  misc_feature (31)..(31): n is g or a
cctgtgggcagtcataatctcaaggcggccnccaaggccaagctaggctatgatcccgtggag

SEQ ID NO: 6 - DNA, 63 bp, Homo sapiens
  misc_feature (31)..(31): n is g or a
gagcccagcggctacacggtgcgcgaggccngcccgccggcattctacaggccaaattcagat

SEQ ID NO: 7 - DNA, 63 bp, Homo sapiens
  misc_feature (32)..(32): n is t or c
atcccctcacagcgcagggcatccgtctttgngctggacactgtgcgcagcaaccagatgccc

SEQ ID NO: 8 - DNA, 63 bp, Homo sapiens
  misc_feature (31)..(31): n is c or t
cggctgctggacattgacgagacagagtacnatgcagatgggggcaaggtgcccatcaagtgg

SEQ ID NO: 9 - DNA, 63 bp, Homo sapiens
  misc_feature (33)..(33): n is g or a
cgagaagtgacaggctatgtcctcgtggccatnaatgaattctctactctaccattgcccaac

SEQ ID NO: 10 - DNA, 63 bp, Homo sapiens
  misc_feature (32)..(32): n is g or a
attccatcacaacaagatattctgcttgccanaattccatcacaacaagatattctgcttgcc

SEQ ID NO: 11 - DNA, 73 bp, Homo sapiens
  misc_feature (32)..(43): “TTGGGGGCCGGC” may or may not be present
cggactcaacctctactgtgggggggccggcttgggggccggcagcggcggcgccacccgcccgggagggcga

SEQ ID NO: 12 - DNA, 63 bp, Homo sapiens
  misc_feature (32)..(32): n is g or a
cagtcacagcacatgacggaggttgtgaggcnctgcccccaccatgagcgctgctcagatagc

SEQ ID NO: 13 - protein, 21 aa, Homo sapiens
  misc_feature (11)..(11): X is Y or F
ARLLEGDEKEXNADGGKMPIK

SEQ ID NO: 14 - protein, 21 aa, Homo sapiens
  misc_feature (11)..(11): X is S or F
CRADEFLCNNXLCKLHFWVCD

SEQ ID NO: 15 - protein, 21 aa, Homo sapiens
  misc_feature (11)..(11): X is Q or H
KNPAERADLKXLMVHAFIKRS

SEQ ID NO: 16 - protein, 21 aa, Homo sapiens
  misc_feature (11)..(11): X is T or I
HPAIRMAPSLXFKEKVTSLKF

SEQ ID NO: 17 - protein, 21 aa, Homo sapiens
  misc_feature (11)..(11): X is A or T
PVGSHNLKAAXKAKLGYDPVE

SEQ ID NO: 18 - protein, 21 aa, Homo sapiens
  misc_feature (11)..(11): X is G or S
EPSGYTVREAXPPAFYRPNSD

SEQ ID NO: 19 - protein, 21 aa, Homo sapiens
  misc_feature (11)..(11): X is V or A
IPSQRRASVFXLDTVRSNQMP

SEQ ID NO: 20 - protein, 20 aa, Homo sapiens
  misc_feature (10)..(10): X is H or Y
LLDIDETEYXADGGKVPIKW

SEQ ID NO: 21 - protein, 21 aa, Homo sapiens
  misc_feature (11)..(11): X is M or I
REVTGYVLVAXNEFSTLPLPN

SEQ ID NO: 22 - protein, 21 aa, Homo sapiens
  misc_feature (11)..(11): X is R or K
IPSQQDILLAXRPTKGIHEYD

SEQ ID NO: 23 - protein, 25 aa, Homo sapiens
  SITE (12)..(15): “Leu Gly Ala Gly” may or may not be present
IGLNLYCGGAGLGAGSGGATRPGGR

SEQ ID NO: 24 - protein, 21 aa, Homo sapiens
  misc_feature (11)..(11): X is R or H
QSQHMTEVVRXCPHHERCSDS

SEQ ID NO: 25 - protein, 10 aa, Homo sapiens
RLLEGDEKEF

SEQ ID NO: 26 - protein, 10 aa, Homo sapiens
RLLEGDEKEY

SEQ ID NO: 27 - protein, 14 aa, Homo sapiens
FLCNNFLCKLHFWV

SEQ ID NO: 28 - protein, 14 aa, Homo sapiens
FLCNNSLCKLHFWV

SEQ ID NO: 29 - protein, 8 aa, Homo sapiens
HLMVHAFI

SEQ ID NO: 30 - protein, 8 aa, Homo sapiens
QLMVHAFI

SEQ ID NO: 31 - protein, 11 aa, Homo sapiens
SLIFKEKVTSL

SEQ ID NO: 32 - protein, 11 aa, Homo sapiens
SLTFKEKVTSL

SEQ ID NO: 33 - protein, 10 aa, Homo sapiens
NLKAATKAKL

SEQ ID NO: 34 - protein, 10 aa, Homo sapiens
NLKAAAKAKL

SEQ ID NO: 35 - protein, 10 aa, Homo sapiens
REASPPAFYR

SEQ ID NO: 36 - protein, 10 aa, Homo sapiens
REAGPPAFYR

SEQ ID NO: 37 - protein, 9 aa, Homo sapiens
SVFALDTVR

SEQ ID NO: 38 - protein, 9 aa, Homo sapiens
SVFVLDTVR

SEQ ID NO: 39 - protein, 9 aa, Homo sapiens
YADGGKVPI

SEQ ID NO: 40 - protein, 9 aa, Homo sapiens
HADGGKVPI

SEQ ID NO: 41 - protein, 9 aa, Homo sapiens
YVLVAINEF

SEQ ID NO: 42 - protein, 9 aa, Homo sapiens
YVLVAMNEF

SEQ ID NO: 43 - protein, 10 aa, Homo sapiens
LLAKRPTKGI

SEQ ID NO: 44 - protein, 10 aa, Homo sapiens
LLARRPTKGI

SEQ ID NO: 45 - protein, 9 aa, Homo sapiens
NLYCGGAGS

SEQ ID NO: 46 - protein, 13 aa, Homo sapiens
NLYCGGAGLGAGS

SEQ ID NO: 47 - protein, 9 aa, Homo sapiens
HMTEVVRHC

SEQ ID NO: 48 - protein, 9 aa, Homo sapiens
HMTEVVRRC

SEQ ID NO: 49 - protein, 21 aa, Homo sapiens
SGWVKPIIIGHHAYGDQYRAT

SEQ ID NO: 50 - protein, 21 aa, Homo sapiens
SGWVKPIIIGGHAYGDQYRAT

SEQ ID NO: 51 - protein, 21 aa, Homo sapiens
VNPEGKYSFGVTCVKKCPRNY

SEQ ID NO: 52 - protein, 21 aa, Homo sapiens
VNPEGKYSFGTTCVKKCPRNY

SEQ ID NO: 53 - protein, 21 aa, Homo sapiens
STRDPLSEITKQEKDFLWSHR

SEQ ID NO: 54 - protein, 21 aa, Homo sapiens
STRDPLSEITGQEKDFLWSHR

SEQ ID NO: 55 - protein, 21 aa, Homo sapiens
EYFMKQMNDARHGGWTTKMDW

SEQ ID NO: 56 - protein, 19 aa, Homo sapiens
EYKLVVVGADGVGKSALTI

SEQ ID NO: 57 - protein, 19 aa, Homo sapiens
EYKLVVVGAVGVGKSALTI

SEQ ID NO: 58 - protein, 19 aa, Homo sapiens
EYKLVVVGACGVGKSALTI

SEQ ID NO: 59 - protein, 21 aa, Homo sapiens
QSQHMTEVVRHCPHHERCSDS

SEQ ID NO: 60 - protein, 21 aa, Homo sapiens
NLLGRNSFEVCVCACPGRDRR

SEQ ID NO: 61 - protein, 21 aa, Homo sapiens
NLLGRNSFEVHVCACPGRDRR

SEQ ID NO: 62 - protein, 21 aa, Homo sapiens
ARLLEGDEKEFNADGGKMPIK

SEQ ID NO: 63 - protein, 21 aa, Homo sapiens
KNPAERADLKHLMVHAFIKRS

SEQ ID NO: 64 - protein, 21 aa, Homo sapiens
HPAIRMAPSLIFKEKVTSLKF

SEQ ID NO: 65 - protein, 21 aa, Homo sapiens
PVGSHNLKAATKAKLGYDPVE

SEQ ID NO: 66 - protein, 21 aa, Homo sapiens
EPSGYTVREASPPAFYRPNSD

SEQ ID NO: 67 - protein, 21 aa, Homo sapiens
VPSDQDLLRCHVLTSGIFETK

SEQ ID NO: 68 - protein, 21 aa, Homo sapiens
SMKCKNVVPLNDLLLEMLDAH

SEQ ID NO: 69 - protein, 21 aa, Homo sapiens
RGTGLEENNQKEQSMDSNLGE

SEQ ID NO: 70 - protein, 21 aa, Homo sapiens
VNGWTGEDCSKNIDDCASAAC

SEQ ID NO: 71 - protein, 19 aa, Homo sapiens
MQPDPRPSRAGACCRFLPL

(19) United States
(12) Patent Application Publication
(10) Pub. No.: US 2023/0323431 A1
(43) Pub. Date: Oct. 12, 2023

(54) Personalized Immunogenic Compositions and Methods for Producing and Using Same

(71) Applicant: World Biotech Regenerative Medical Group Limited, Hong Kong SAR (CN)

(72) Inventors: Wing Yin Cornelia MAN, Hong Kong SAR (CN); Wei MA, Hong Kong SAR (CN); Quangui GAO, Hong Kong SAR (CN)

(21) Appl. No.: 18/023,621

(22) PCT Filed: Aug. 31, 2021
(86) PCT No.: PCT/IB2021/057969

§ 371 Date: Feb. 27, 2023

Related U.S. Application Data
(60) Provisional application No. 63/072,913, filed on Aug. 31, 2020.

(51) Int. Cl. C12Q 1/6827; C07K 16/28

(52) U.S. Cl. CPC C12Q 1/6827; C07K 16/2833; A61K 2039/6006

(57) ABSTRACT

Provided is a method of preparing a personalized immunogenic composition, which may be prepared by obtaining genetic sequences from a liquid biopsy, comparing the genetic sequences to a wild-type reference genome to identify mutant sequences, selecting epitopes from the mutant sequences, producing the peptides encoded by the selected epitopes, and incorporating the produced peptides into an immunogenic composition. Obtaining the genetic sequences may include next-generation sequencing of genetic material that has been enriched from the liquid biopsy. Deep sequencing (average coverage of 10,000× and above) may be used to detect gene mutations with rare frequencies. Immunogenicity of selected epitopes may be predicted using various in silico methods and epitopes used in an immunogenic composition may be selected from those selected epitopes with high binding amity to HLA. An immunogenic composition prepared using these methods may be administered to a subject.

### CROSS REFERENCE TO RELATED APPLICATIONS

This application claims priority under 35 U.S.C. § 119(e) to U.S. Provisional Application No. 63/072,913, filed on Aug. 31, 2020, which is hereby incorporated by reference in its entirety.

### FIELD OF THE DISCLOSURE

The present disclosure relates, in some embodiments, to personalized immunogenic compositions and methods for producing and using them.

### BACKGROUND OF THE DISCLOSURE

Cancer is the second leading cause of death globally. For early stage cancers that are non-metastatic, surgical removal can be an effective treatment. However, for more advanced and refractory cases, chemotherapies, target therapies, and radiotherapies are often used. Such therapies may extend survival and reduce symptoms, but may not lead to complete remission. Resistance development is common and diseases often relapse after initial treatments. Immuno-checkpoint therapies such as PD-1 blocking antibodies have been shown to be effective in a subset of cancers by re-activating the patient's own immune system to fight against the cancer cells. However, due to the high heterogeneity nature of cancer cells, a majority of patients still respond sub-optimally to such immunotherapies. A critical mechanism by which tumors evade immune surveillance is by local downregulation of cancer-specific T-cells. Any gene products expressed differently (in mutated forms) in cancer cells compared to normal cells are potential neoantigens.

The idea of developing personalized immunogenic compositions has been a goal for many decades. Acquisition of multiple somatic mutations is a hallmark of cancer and is essential for healthy cells' transformation to cancerous cells. Such mutations generate cancer-associated neoantigens that may be targets of the adaptive immune system. Cancer patients, and even healthy subjects, do have unique or unique sets of neoantigens.

By artificially administering the neoantigens to subjects, we can activate and induce expansion of the subject's endogenous “tumor-specific lymphocytes”. The subject's body then recognizes the mutant antigens expressed by cancer cells and implements the killing of tumor cells.

Unlike traditional chemo/targeted therapies, an immunogenic composition includes the generation of immunological memory that contributes to prevention of cancer recurrence. Accordingly, there is a strong demand for immunogenic compositions having strong immunogenicity. That is, immunogenic compositions which may turn immunologically cold tumour(s) into tumor(s) with higher mutational burdens.

Despite being a promising opportunity, the generation of immunogenic compositions faces many challenges including identification of cancer-associated neoantigens and translation of such neoantigens into vaccines. The present disclosure is drawn to methods of preparing an immunogenic composition configured to prime the human immune system to generate antibodies targeted to neoantigens, such immunogenic compositions serving as personalized/customized immunogenic compositions for both prevention and therapeutic treatments of cancers.

The present disclosure is further drawn to the compositions generated by such methods.

### SUMMARY

A method of preparing an immunogenic composition configured to prime the human immune system to generate stimulatory response in cancer-specific T cells targeted to neoantigens may include: determining target genetic sequences from genetic sequences present in a liquid biopsy (e.g., peripheral blood) obtained from a subject; comparing the target genetic sequences to a reference sequence comprising a wild-type genetic sequence to identify mutant genetic sequences having one or more non-synonymous mutations; selecting one or more potential epitopes from the mutant genetic sequences; identifying a confirmed epitope based on an immunogenicity of the one or more potential epitopes; producing a mutant peptide comprising the confirmed epitope; and combining the mutant peptide with a carrier or an immunostimulant to form the immunogenic composition.

Determining target genetic sequences may include enrichment of one or more types of genetic material present in a liquid biopsy. Enrichment may include any one or more of: applying positive selection based on cell size and surface protein marker expression; applying negative selection based on removal of white blood cells using antibody-coated magnetic beads; silica-based DNA capture methods; and carboxyl-modified-group-based DNA capture methods.

In some embodiments, determining target genetic sequences may comprise: enriching the liquid biopsy for circulating tumor cells (CTC) and cell-free DNA (cfDNA); extracting circulating tumor DNA (ctDNA) from the enriched CTC; determining a genetic sequence for each of the ctDNA, the cell-free DNA (cfDNA), and exosomal DNA present in the liquid biopsy. Determining the genetic sequence may include using next-generation sequencing technology. Determining the genetic sequence may further include using deep sequencing comprising an average coverage of at least 10,000×.

Target genetic sequences, according to some embodiments, may comprise any one or more of: ctDNA, cfDNA, exosomal DNA, enriched cfDNA, and DNA extracted from enriched CTC.

Selecting one or more potential epitopes, according to some embodiments, may include removing germline mutations from the mutant genetic sequences, which may include: comparing the mutant genetic sequences to peripheral blood mononuclear cell (PBMC) sequences from the subject; identifying the germline mutations, where the germline mutations include sequences that are present in both the mutant genetic sequences and the PBMC sequences; and removing the germline mutations from the mutant genetic sequences.

Identifying a confirmed epitope, according to some embodiments, may include determining the subject's human leukocyte antigen (HLA) class type; and determining binding affinities for the potential epitopes, wherein the binding affinities are based on the subject's HLA class type. Determining the subject's HLA class type may include using one or more of sequence-specific primer PCR, real time qPCR and next-generation sequencing.

Determining binding affinities for the mutant genetic sequences may include: determining a resulting peptide sequence encoded within the genetic sequences of the potential epitopes, using a computer-based algorithm for predicting half-maximal inhibitory concentration (IC50) values for the resulting peptide sequence binding to the HLA sequence of the subject, and selecting confirmed epitopes from those potential epitopes with IC50 values eliciting efficacy to provoke cancer-specific T-cell immune responses.

In some embodiments, identifying a confirmed epitope may further include: determining a resulting peptide sequence encoded within the wild-type genetic sequence corresponding to the potential epitopes, using a computer-based algorithm for predicting IC50 values for the resulting wild-type peptide sequence binding to HLA of the subject, and removing confirmed epitopes whose corresponding wild-type peptide sequences have high IC50 values. Identifying a confirmed epitope may include measuring endogenous cytotoxic T cell (CTL) activation by the confirmed epitope by determining the level of interferon gamma (IFNγ) secretion using one or more of an ELISpot assay; a high throughput screening ELISA assay; and intracellular cytokine flow cytometry targeting interleukin 2, tumor necrosis factor-alpha, and IFNγ. Identifying a confirmed epitope may include measuring endogenous HLA sequences and the confirmed epitope containing anchor positions & form Ternary complexes with a specific repertoire of T-Cell Receptors on Cytotoxic T Lymphocytes (CTLs).

A carrier may include autologous DC, which may include expanded monocytes isolated from the subject's PBMC.

A method may further include administering the immunogenic composition to the subject.

### DETAILED DESCRIPTION

The present disclosure relates to methods for identifying, predicting and selecting cancer specific mutations for the synthesis of mutant peptides to produce personalized immunogenic compositions configured to prime the human immune system to generate antibodies targeted to neoantigens. In some embodiments, liquid biopsy from subjects may provide information about the mutation status of subjects without the need of a tissue biopsy. In some embodiments, a method may include drawing peripheral blood from subjects and may include enriching DNA from CTC, cfDNA, ctDNA, and/or exosomes. The sequence of CTC DNA or cfDNA or ctDNA or exosomal DNA from subjects may be used to identify cancer specific mutations, predict mutant immunogenicity and produce personalized immunogenic compositions.

A method may include identifying mutant sequences which code for all or a portion of a gene as those which have a mutant amino acid substituting for a wild-type amino acid located at the same position in the wild-type sequence of the protein.

Also provided herein are mutant DNA sequences associated with cancer and mutant peptide sequences predicted to be immunogenic.

Additionally provided is a personalized immunogenic composition comprising at least one mutant peptide, subject's autologous DC, and/or adjuvants, and methods to prepare the same. In some embodiments, a method may include synthesizing immunogenic mutant peptides under GMP condition. A subject's own DC cells may be expanded from their PBMC under GMP condition. Expanded DC cells may be loaded with the synthesized mutant peptides and mixed with adjuvants.

Further provided are methods of immunizing a subject by identifying immunogenic cancer specific antigens from DNA from CTC, cfDNA, ctDNA, or exosomal DNA as described by any of the methods herein and by preparing a vaccine with one or more of the selected mutant antigens. A method may include immunizing a subject by administering a vaccine. A vaccine of the present disclosure may immunize the subject against the mutant antigens and activates the subject's own CTL specific to the mutant antigens to kill cancer cells expressing the mutant antigens while not damaging normal cells without the mutant antigens. An activation and/or clonal expansion of the subject's mutant specific CTL can be monitored using intracellular cytokine flow cytometry.

Provided herein are methods to identify gene mutations which generate immunogenic neoantigens in cancer cells from subjects and to use the antigens to load expanded autologous DC cells from the subjects to produce cell vaccines. Subjects may be immunized by administrating an effective dose of the vaccines to activate their own mutant peptide specific T cells for killing of the cancer cells.

### Methods of Preparing an Immunogenic Composition

In some embodiments, a method of preparing an immunogenic composition may include: determining target genetic sequences including genetic sequences present in a liquid biopsy obtained from a subject; comparing the target genetic sequences to a reference sequence including a wild-type genetic sequence to identify mutant genetic sequences including one or more non-synonymous mutations; selecting one or more potential epitopes from the mutant genetic sequences; identifying a confirmed epitope based on an immunogenicity of the one or more potential epitopes; producing a mutant peptide including the confirmed epitope; and combining the mutant peptide with a carrier to form the immunogenic composition.

### Determining Target Genetic Sequences

Liquid biopsy from a subject may be used as a sample to detect cancer associated gene mutations and to determine target genetic sequences. A subject may be any animal including, in some embodiments, a human. In some embodiments, a liquid biopsy may be a fluid (e.g., blood, peripheral blood, cerebrospinal fluid, lymphatic fluid, plasma, urine, aspirate, etc.). As used herein, the term “liquid biopsy” may encompass tests done on subject's fluid (e.g., peripheral blood) to look for cancer cells from a tumor that are circulating in the blood and/or for DNA fragments from cancer cells that are released to the blood. Liquid biopsy has the advantage to detect tumor-released materials in early stages and in tumors which tissue biopsy cannot be obtained. It is a non-invasive and safe method to obtain materials of cancer cells. This enables the disclosed technologies to be applicable to the general public.

A method may include drawing fluid (e.g., blood, peripheral blood, etc.) from the subject. Where a fluid includes blood (e.g., peripheral blood, etc.), a method may include mixing the blood with anticoagulant.

In some embodiments, determining target genetic sequences may include enrichment of one or more types of genetic material present in a liquid biopsy (e.g., fluid). While it should be understood that many types (e.g., sources) of genetic material may be enriched, in some embodiments, enrichment of one or more of CTC, ctDNA, cfDNA, and exosomal DNA may be performed. DNA that has been isolated from CTC (e.g., enriched CTC) may be referred to as ctDNA. Enrichment of genetic material may include any enrichment technique, including amplification, positive selection, and negative selection. For example, in some embodiments, enrichment of one or more of cfDNA, ctDNA, and exosomal DNA may be achieved by using any number of DNA selection techniques, including silica-based DNA capture methods, carboxyl-modified-group-based DNA capture methods, or both.

In some embodiments, enrichment may be performed at the cellular level and may include positive selection, negative selection, or both. For example, the enrichment of CTC may be performed by applying positive selection. Positive selection may include those types of enrichment based on cell size and surface protein marker expression. In some embodiments, enrichment of negative selection based on removal of white blood cells. In a specific embodiment, negative selection may include using antibody-coated magnetic beads. DNA may be isolated from CTC (e.g., enriched CTC) and, according to some embodiments, may be further enriched using DNA selection techniques.

Determining target genetic sequences may include amplification of genetic material present in a liquid biopsy. Any number of DNA amplification techniques (e.g., PCR, RTPCR, qPCR, etc.) may be used. A method may include sequencing genetic material present in a liquid biopsy. In some embodiments, a method may include: sequencing genetic material from an enriched sample (e.g., enriched for CTC, ctDNA, cfDNA, exosomal DNA), sequencing of amplified (e.g., PCR) genetic material, or any combination thereof. In some embodiments, sequencing of genetic material may include next generation sequencing (NGS). A method may include, in some embodiments: sequencing the genome of normal (e.g., non-cancerous) cells from the subject, sequencing the subject's peripheral blood mononuclear cell genome (PBMC genome) using ultra-deep NGS sequencing methods, or both.

A method may include preparing a genomic library by selecting particular sequences using any one or more of the following target-enrichment methods: hybrid capture, in-solution capture, and PCR amplicon amplification. A genomic library may include sequences from exons, introns, promoters and non-coding sequences. In some embodiments, a genomic library may include the exon sequences which contain the protein coding sequences. Any sequences which may lead to generation of immunogenic mutant peptides can be included in the genomic library.

In some embodiments, a method may include adding unique molecular identifiers (UMI) to the genomic library. UMI are DNA barcodes that are ligated to each single DNA fragments at the beginning steps of library preparation. By sequencing the UMI, one can identify the original DNA fragments of each sequences produced in the final NGS result. A method may include grouping sequences coming from the same UMI, and may include generating a consensus sequence of the original DNA fragment with lower error rate. This lowers the noises in the NGS results and enable identification of gene mutations with low frequencies.

A method may include obtaining sequences of the libraries by applying NGS to each of the libraries. NGS sequencing can be done using sequencers (e.g., next-generation sequencers). In some embodiments, target genetic sequences may include the sequences obtained from sequencing any one or more of cfDNA, ctDNA, exosomal DNA, PBMC genome, and the genome of normal (e.g., non-cancerous) cells from a subject (e.g., a subject's liquid biopsy).

### Identifying Mutant Genetic Sequences and Selecting Potential Epitopes

A method may include identifying mutant sequences which contain one or more non-synonymous mutation. In some embodiments, identifying mutant genetic sequences including one or more non-synonymous mutations may include comparing the target genetic sequences to a reference sequence. A reference sequence may include one or more wild-type genetic sequences and, in a specific embodiment, may include a wild-type genetic sequence from a human. Identified mutations (e.g., mutant genetic sequences) may result in a substitution of a wild-type amino acid but may also result in amino acid changes caused by deletions or insertions of nucleotide sequence in the encoding nucleic acid.

In some embodiments, a method may include deep sequencing. Deep sequencing differs from conventional NGS sequencing methods which normally produce coverage around 20×. The high coverage of deep sequencing enables discovering mutant sequences with rare Variant Allele Frequencies (VAF) (e.g. less than 0.5%) which are not detectable by current common NGS methods. Deep sequencing may include a high number of unique reads for each region and/or given nucleotide of a sequence (e.g., high coverage). For example, in some embodiments, deep sequencing may include coverage of greater than about 8000 reads, or greater than about 8500 reads, or greater than about 9000 reads, or greater than about 9500 reads, or greater than about 10,000 reads, or greater than about 10,500 reads, where about means plus or minus 250 reads. In some embodiments, deep sequencing may be performed (in some embodiments, with average coverage of 10,000× and above) to enable detection of gene mutations with rare frequencies when target genetic sequences are compared (e.g., mapped) to a reference sequence.

A method may include selecting potential epitopes from mutant genetic sequences. As noted above, mutant genetic sequences may be identified by mapping (e.g., comparing) target genetic sequences to a reference sequence to identify non-synonymous mutations. Mutant genetic sequences may include germline mutations which may, according to some embodiments, not be suitable epitopes for use in an immunogenic composition. Therefore, identification of germline mutations may be preferred. To remove germline mutations and identify potential epitopes, target genetic sequences may be compared to the sequences of normal cells (e.g., PBMC) from the same subject. Potential epitopes, according to some embodiments, may be those which sequences which are present in the mutant genetic sequences and are absent in the DNA sequences from normal cells (e.g. PBMC) from the subject.

### Identifying Confirmed Epitopes

A method may include identifying one or more confirmed epitopes, based on the immunogenicity of the one or more potential epitopes. In some embodiments, this may include a further selection of and refinement of potential epitopes selected from the mutant genetic sequences. In some embodiments, identification of confirmed epitopes from the large number of mutant sequences identified by NGS analysis may depend on the expressed major histocompatibility complex (MHC) class I or class II super type of the subject. The subject's MHC super types determine whether the potential epitope can be presented by their own DC cells and whether the potential epitope is able to bind to cancer specific T-cell Receptors (CTRs) to activate their own Cytotoxic T cells (CTLs). Identifying confirmed epitopes may include typing (e.g., identifying) a subject's HLA class type. The subject's MHC class I or class II type may be typed, according to some embodiments, using sequence-specific primer PCR, real time qPCR or NGS.

A method may include selecting confirmed epitopes from a large number of potential epitopes by evaluation and ranking based on the ability of a given potential epitope to bind to human leukocyte antigen (HLA) complexes of the same subject. In some embodiments, in silico algorithm(s) may be used for predicting a potential epitope's affinity against the subject's HLA. In some embodiments, the ability of the potential epitopes to be recognized by endogenous CTL may be carried out by synthesizing labeled peptide-HLA complex and testing them for binding to endogenous CTL at the cancer-specific CTRs in PBMC. In some embodiments, activation of CTLs by the mutant peptides may be elevated using cytokine detecting immunoassays. In some embodiments, humanized mice may be used to confirm the immunogenicity of the mutant peptides in vivo.

A method may include determining the amino acid sequence (e.g., resulting peptide) coded by the nucleotide sequence of one or more potential epitopes. Selection of confirmed epitopes from potential epitopes that may be suitable for HLA presentation, according to some embodiments, may be carried out in silico using computer-based algorithm(s) for predicting IC50 values for resulting peptides (e.g., those resulting peptides coded by a potential epitope's nucleotide sequence) binding to the subject's specific HLA molecules. Any number of prediction tools may be used to predict potential epitope resulting peptide binding affinities to HLA. A method may include evaluating the binding epitopes of one or more potential epitopes, using the difference between the mutant's (e.g., potential epitope) affinity and the wild-type's affinity. A rank list may be produced to indicate one or more high-affinity epitopes. In some embodiments, confirmed epitopes may include those potential epitopes with high HLA affinity.

In some embodiments, a method may include predicting HLA affinity for a wild-type peptide. For example, where a potential epitope has been identified as a candidate for a confirmed epitope (e.g., one to be used in an immunogenic composition), the corresponding wild-type peptide, encoded in the wild-type nucleotide sequence, may be evaluated in silico using computer-based algorithm(s) for predicting IC50 values for the resulting peptide's binding to the subject's specific HLA molecules. In some embodiments, confirmed epitopes may be those potential epitopes having high HLA affinity but with low HLA affinity for the corresponding wild-type peptide.

In some embodiments, confirmed epitopes that are predicted to bind to HLA class I may be further evaluated to determine if they are recognized by the CTRs of the CTLs in the subject's own PBMC. For example, PBMC of the subject can be stained using fluorochrome labeled confirmed epitope-HLA complex and analyzed by flow cytometry. A method may include, in some embodiments, measuring activation of CTLs by determining the level of IFNγ secretion using an ELISpot assay, or a high throughput screening ELISA assay. Intracellular cytokine flow cytometry targeting interleukin 2, tumor necrosis factor-alpha and IFNγ can also be performed to determine the activation responses of CTLs.

In some embodiments, a humanized mice model may be used to further validate the immunogenicity of an epitope (e.g., potential epitope, confirmed epitope). A method may include injecting human hematopoietic stem cells to irradiated mice to replace their immune system with a humanized one. Mutant peptides (e.g., potential epitope, confirmed epitope) may be injected intravenously into the humanized mice. The PBMC may be extracted from those injected mice. Flow cytometry using fluorochrome labeled mutant peptide-HLA complex may be performed to measure the expansion of antigen specific T cells. A method may include performing intracellular cytokine flow cytometry to measure the level of activation of those antigen specific T cells. In some embodiments, a method may further confirm the immunogenicity of one or more mutant peptides (e.g., potential epitope, confirmed epitope) in vivo.

In some embodiments, the above experimental elevation of mutant peptides' immunogenicity may be critical for the selection of mutant peptides that are optimally effective in the subject. Comparing to methods which only elevate mutant peptide immunogenicity by in silico analysis alone, the present disclosure, which employs the subject's own immune cells to confirm the selected mutant peptides, is capable to provoke significant immune response by activating endogenous CTLs.

### Forming an Immunogenic Composition

Subjects with risk of developing cancer(s) and with diagnosed cancer(s) may benefit from this method for the development of personalized immunogenic compositions. Cancers suitable for analysis may include (but are not limited to) carcinomas, sarcomas, leukemias, and lymphomas. Carcinomas may be of breast, colon, rectum, lung, oropharynx, hypopharynx, esophagus, stomach, pancreas, liver, gallbladder and bile ducts, small intestine, urinary tract, female genital tract, male genital tract, endocrine glands, and skin in nature. Other cancers including hemangiomas, meningiomas, melanomas, and tumors of the brain, nerves, and eyes can also be included.

Preparing an immunogenic composition (e.g., a personalized immunogenic composition) may include one or a mixture of different peptide sequences including confirmed epitopes identified using the methods above. The peptides (e.g., confirmed epitopes) may be prepared by peptide synthesis chemistry under good manufacturing practice (GMP) conditions.

A method may include forming an immunogenic composition using peptides including one or more confirmed epitopes recognized by subject's HLA and predicted to be immunogenic. Such immunogenic composition may be administered to an individual in order to activate mutant specific CTLs. The activated CTLs may expand and specifically recognize the mutant peptide expressed by cancer cells which eventually leads to cancer cell killing.

An immunogenic composition may contain at least one confirmed epitope or multiple confirmed epitopes, such as 2, 3, 4, 5 or more. The confirmed epitopes that are used in the vaccine may be chosen according to their ability to bind to HLA antigens expressed by the subject who is to receive the vaccine. They may be peptides that can be recognized and bind to the subject's endogenous CTRs and have the ability to activate the subject's CTLs.

In some embodiments, a personalized immunogenic composition can include a carrier. In some embodiments, a carrier may enhance the resulting immune response to the peptide including the confirmed epitope. A carrier may include a carrier protein, which may be selected from any carrier suitable for use in an immunogenic composition (e.g., Tetanus toxoid, Diphtheria toxin, membrane associated proteins, B. pertussis fimbriae, etc.).

In some embodiments, a carrier may include a carrier protein specific to a subject, such as the subject's autologous dendritic cells (autologous DC). Autologous DC may be expanded using monocytes isolated from the subject's PBMC. Isolated monocytes may be expanded ex vivo and a combination of cytokines may be added to the culture medium to induce them to differentiate into DC. The whole process may be done under GMP conditions.

In some embodiments an immunogenic composition may include an adjuvant. An adjuvant may be used to augment the effects of a vaccine by stimulating the immune system. Adjuvants can include one or more of aluminum salts, liposomes, lipopolysaccharide, polyinosinic: polycytidylic acid, interleukins (e.g., IL-12), unmethylated CpG dinucleotide DNA, and other adjuvant materials.

### Methods of Using an Immunogenic Composition

A method of immunizing a subject against mutant peptide expressing cancer cells may include administering a personalized immunogenic composition including one or more mutant peptides including one or more confirmed epitopes. In some embodiments, an immunogenic composition may be a loaded DC vaccine (e.g., a DC vaccine containing confirmed epitopes).

An immunogenic composition can be administered in a sufficient amount to treat a subject that has cancer cells expressing the mutant peptides including the confirmed epitopes. An administered vaccine may activate mutant peptide specific CTLs and/or trigger clonal expansion of the mutant peptide specific CTLs. The activated CTLs kill the cancer cells thereby treating the subject. Immunological memory may be formed thereby protecting the subject from developing mutant peptide expressing cancers in the long run.

A method may include determining a physiologically effective dose. A physiologically effective dose can be estimated initially from cancer cell culture cytotoxic assays by determining an IC50. IC50 is the concentration of the immunogenic composition required to bring the dose-response curve down by half. An efficacious dose is determined by IC50 value or values eliciting or provoking cancer-specific T-cell immune responses. In cancer cell culture, the IC50s of the immunogenic compositions selected from the peptide library may range from 10 μg to 50 μg. A dose can then be translated in animal models to validate the IC50 as determined in cell cultures. In humanized mice models, the strength & persistence of the cancer specific T-cells activation may be measured. Such information can be used to more accurately determine useful initial doses in human clinical trials. The exact formulation, route of administration and dosage can be chosen by the principle investigator or physician in view of the subject's condition.

Subjects may be immunized again (e.g., administered another dose of an immunogenic composition) by the methods of the disclosure if the subjects show decreased mutant expressing circulating tumour cells (CTCs) after vaccine administration and/or expansion and activation of CTLs specific against the administrated mutant antigens.

Mutant expressing CTCs can be monitored using CTC screening platforms (e.g., CellSearch (Menarini Silicon Biosystems Inc), ClearCell FX1 (Biolidics) and immunofluorescence microscopy) Expansion and activation of mutant peptide specific CTLs can be monitored using flow cytometry.

In addition, a method may include immunizing a subject and preventing a subject from having cancer. Cancer prevention, according to some embodiments, may be the absence of mutant-expressing cancer development after the vaccine administration with no evidence of disease as indicated by diagnostic methods, such as imaging, such as PET/CT and MRI.

### EXAMPLES

### Example 1: Identifying Cancer Specific Mutations from Genomic Libraries Using NGS

In this example, serum and PBMC were isolated from subject's peripheral blood. cfDNA and exosomal DNA were isolated from serum using silica-based gel columns. CTC was enriched from PBMC using magnetic beads based CD45 negative selection. Following CTC enrichment, DNA was extracted from the CTC. DNA from PBMC is extracted as control.

| SEQ | | | | |
|---|---|---|---|---|
| ID | | | | DNA sequence with mutant |
| NO | Gene | RefSeq | Variant | nucleotide marked in blankets |
| 1 | ERBB4 | NM 005235.3 | c2624A>T | GCCAGACTCTTGGAAGGAGATGAAAAAGAGT |
| | | | | (A/T)CAATGCTGATGGAGGAAAGATGCCAA |
| | | | | TTAAA |
| 2 | LRP1B | NM_018557.3 | c11051C>T | TGTAGAGCTGATGAGTTCCTTTGCAATAATT |
| | | | | (C/T)TCTCTGCAAACTACATTTCTGGGTGT |
| | | | | GTGAT |
| 3 | MAP2K1 | NM_002755.4 | c1062A>C | AAAAACCCCGCAGAGAGAGCAGATTTGAAGC |
| | | | | A(A/C)CTCATGGTTCATGCTTTTATCAAGA |
| | | | | GATCT |
| 4 | NF1 | NM_001042492.3 | c.1400C>T | CACCCAGCAATACGAATGGCACCGAGTCTTA |
| | | | | (C/T)ATTTAAAGAAAAAGTAACAAGCCTTA |
| | | | | AATTT |
| 5 | POLE | NM_006231.3 | c.1282G>A | CCTGTGGGCAGTCATAATCTCAAGGCGGCC |
| | | | | (G/A)CCAAGGCCAAGCTAGGCTATGATCC |
| | | | | CGTGGAG |
| 6 | ESR1 | NM_001291230.1 | c.433G>A | GAGCCCAGCGGCTACACGGTGCGCGAGGCC |
| | | | | (G/A)GCCCGCCGGCATTCTACAGGCCAAA |
| | | | | TTCAGAT |
| 7 | POLE | NM_006231.3 | c.4541T>C | ATCCCCTCACAGCGCAGGGCATCCGTCTTTG |
| | | | | (T/C)GCTGGACACTGTGCGCAGCAACCAGA |
| | | | | TGCCC |
| 8 | ERBB2 | NM_004448.4 | c.2632C>T | CGGCTGCTGGACATTGACGAGACAGAGTAC |
| | | | | (C/T)AT GCAGATGGGGGCAAGGTGCCCA |
| | | | | TCAAGTGG |
| 9 | ERBB3 | NM_001982 | c.93G>A | CGAGAAGTGACAGGCTATGTCCTCGTGGCCA |
| | | | | T(G/A)AATGAATTCTCTACTCTACCATTGC |
| | | | | CCAAC |
| 10 | GNA13 | NM_006572.6 | c.599G>A | ATTCCATCACAACAAGATATTCTGCTTGCCA |
| | | | | (G/A)AATTCCATCACAACAAGATATTCTGC |
| | | | | TTGCC |
| 11 | MCL1 | NM_021960.5 | c.61_72del | CGGACTCAACCTCTACTGTGGGGGGGCCGGC |
| | | | | (TTGGGGGCCGGC/*)AGCGGCGGCGCCACC |
| | | | | CGCCCGGGAGGGCGA |
| 12 | TP53 | NM_000546.6 | c.524G>A | CAGTCACAGCACATGACGGAGGTTGTGAGGC |
| | | | | (G/A)CTGCCCCCACCATGAGCGCTGCTCAG |
| | | | | ATAGC |

DNA libraries were prepared from CTC DNA, cfDNA, exosomal DNA, and PBMC DNA using a PCR amplicon amplification kit. The libraries were checked for their quality using Bioanalyzer. Libraries passing quality check are quantified using real time PCR. Equal amounts of each library was loaded into the sequencer and deep sequenced using NGS platforms.

The sequencing results from each library were initially mapped to the human reference genome NCBI37 hg19 sequence (Genome Bioinformatics Group of the University of California Santa Cruz). The sequences from each cell source which differed from the reference gene sequences resulted in an initial set of potential epitopes.

In this example, the mutation set was further selected using the criteria set below:
1. Discard all variants that locate outside exonic regions;
2. Select sequences where the base difference results in a non-synonymous amino-acid change; and
3. Discard germline mutations identified in the PBMC DNA control library.

The result of these additional selections was applied to the initial mutation set and yielded a smaller set of somatic non-synonymous cancer specific sequences. Results may be found in TABLE 1 and TABLE 2 below. Non-synonymous mutations identified in subjects are referred to below as Set 1 and are presented herein as SEQ ID 1 to SEQ ID 71.

TABLE 1. Results from Example 1, including the nucleotide sequences for potential epitopes identified using the example method.

| SEQ | | | | Peptide sequence | |
|---|---|---|---|---|---|
| ID | | | | with mutant a.a. | |
| NO | Gene | RefSeq | Variant | marked in blankets | Consequence |
| 13 | ERBB4 | NM_005235.3 | c2624A>T | ARLLEGDEKE(Y/F) | substitution |
| | | | | NADGGKMPIK | |
| 14 | LRP1B | NM_018557.3 | c11051C>T | CRADEFLCNN(S/F) | substitution |
| | | | | LCKLHFWVCD | |
| 15 | MAP2K1 | NM_002755.4 | c1062A>C | KNPAERADLK(Q/H) | substitution |
| | | | | LMVHAFIKRS | |
| 16 | NF1 | NM_001042492.3 | c.1400C>T | HPAIRMAPSL(T/D) | substitution |
| | | | | FKEKVTSLKF | |
| 17 | POLE | NM_006231.3 | c.1282G>A | PVGSHNLKAA(A/T) | substitution |
| | | | | KAKLGYDPVE | |
| 18 | ESR1 | NM_001291230.1 | c.433G>A | EPSGYTVREA(G/S) | substitution |
| | | | | PPAFYRPNSD | |
| 19 | POLE | NM_006231.3 | c.4541T>C | IPSQRRASVF(V/A) | substitution |
| | | | | LDTVRSNQMP | |
| 20 | ERBB2 | NM_004448.4 | c.2632C>T | LLDIDETEY(H/Y) | substitution |
| | | | | ADGGKVPIKW | |
| 21 | ERBB3 | NM_001982.4 | c.93G>A | REVTGYVLVA(M/D) | substitution |
| | | | | NEFSTLPLPN | |
| 22 | GNA13 | NM_006572.6 | c.599G>A | IPSQQDILLA(R/K) | substitution |
| | | | | RPTKGIHEYD | |
| 23 | MCL1 | NM_021960.5 | c.61_72del | IGLNLYCGGAG | Inframe |
| | | | | (LGAG/*) | deletion |
| | | | | SGGATRPGGR | |
| 24 | TP53 | NM_000546.6 | c.524G>A | QSQHMTEVVR(R/H) | substitution |
| | | | | CPHHERCSDS | |

TABLE 2. Results from Example 1, including the amino acid sequences for potential epitopes identified using the example method.

### Example 2: Elevation and Validation of Immunogenicity Selected Mutant Peptides

In this example, the potential epitopes of Set 1 were further selected to identify a smaller subset with prospects for binding to HLA antigens of the subject. Subject's HLA was typed using sequence specific primer PCR. Peptide sequences containing the mutant amino acids were transcribed (in silico) as 21mer peptides with amino acids located on each side of the mutant amino acids. The 21mer peptides were then evaluated for having an 8-14 amino acid epitope that would bind to the subject's HLA using the T cell epitope prediction program of IEDB. Peptide sequences were identified that bound below the 10% percentile. The identified peptides were further screened by their difference in predicted binding score between mutant and wild-type. The selected mutant peptides had a higher binding score than the wild-type. The result of these additional selections was applied to Set 1 and, in this example, yielded a smaller set of confirmed epitopes (immunogenic peptides), referred to as Set 2. Results may be found in TABLE 3 below. The peptides in which had high affinity to the subject's own HLA are presented herein as SEQ IDs 1, 3, 4, 8-11, 13, 49, 50, 56-61, and 71.

| | | | | | | Difference |
|---|---|---|---|---|---|---|
| | | | | | | in binding |
| SEQ | | | | | HLA | score |
| ID | | Sequence | Peptide | Peptide | binding | (mutant- |
| NO | Gene | type | length | sequence | score | wildtype) |
| 25 | ERBB4 | mutant | 10 | RLLEGDEKEF | 0.1397 | 0.1240 |
| 26 | | wildtype | 10 | RLLEGDEKEY | 0.0157 | |
| 27 | LRP1B | mutant | 14 | FLCNNFLCKLHFWV | 0.0072 | 0.0004 |
| 28 | | wildtype | 14 | FLCNNSLCKLHFWV | 0.0068 | |
| 29 | MAP2K1 | mutant | 8 | HLMVHAFI | 0.0153 | 0.0081 |
| 30 | | wildtype | 8 | QLMVHAFI | 0.0072 | |
| 31 | NF1 | mutant | 11 | SLIFKEKVTSL | 0.4655 | 0.3947 |
| 32 | | wildtype | | SLTFKEKVTSL | 0.0708 | |
| 33 | POLE | mutant | 10 | NLKAATKAKL | 0.0075 | −0.0005 |
| 34 | | wildtype | 10 | NLKAAAKAKL | 0.0080 | |
| 35 | ESR1 | mutant | 10 | REASPPAFYR | 0.5891 | 0.1591 |
| 36 | | wildtype | 10 | REAGPPAFYR | 0.4300 | |
| 37 | POLE | mutant | 9 | SVFALDTVR | 0.5151 | 0.1920 |
| 38 | | wildtype | 9 | SVFVLDTVR | 0.3231 | |
| 39 | ERBB2 | mutant | 9 | YADGGKVPI | 0.0984 | 0.0879 |
| 40 | | wildtype | 9 | HADGGKVPI | 0.0105 | |
| 41 | ERBB3 | mutant | 9 | YVLVAINEF | 0.0354 | 0.0218 |
| 42 | | wildtype | 9 | YVLVAMNEF | 0.0136 | |
| 43 | GNA13 | mutant | 10 | LLAKRPTKGI | 0.0159 | 0.0093 |
| 44 | | wildtype | 10 | LLARRPTKGI | 0.0066 | |
| 45 | MCL1 | mutant | 9 | NLYCGGAGS | 0.0132 | 0.0124 |
| 16 | | wildtype | 13 | NLYCGGAGLGAGS | 0.0008 | |
| 47 | TP53 | mutant | 9 | HMTEVVRHC | 0.0465 | 0.0103 |
| 48 | | wildtype | 9 | HMTEVVRRC | 0.0362 | |

TABLE 3. Results from Example 2, including the amino acid sequence of confirmed epitopes and relevant binding scores.

### Example 3: Preparation of Personalized Immunogenic Composition

In this example, PBMC was isolated from subject's peripheral blood using density gradient centrifugation. Monocytes were isolated from the PBMC using antibody coated magnetic bead-based selection methods. The isolated monocytes were resuspended in AIM-V medium supplemented with 800 U/ml human GM-CSF and 500 U/ml human IL-4 to induce expansion and differentiation into DC. The cells were incubated in a humidified incubator at 37° C. and 5% CO2 for 7 days. The expanded DC were used to load the selected mutant peptides. The whole process, in this example, was done under GMP condition.

The top ranked mutant peptides in Set 2 were synthesized. The mutant peptides were synthesized by peptide synthesis chemistry under GMP conditions. The expanded autologous DC cells were incubated with sufficient amount of the synthesized mutant peptides in culture to load them with the antigens. The cell number was counted to calculate the concentration and the effective amount to be administrated. Adjuvants were added to the cell mixture and this final vaccine mixture was cryopreserved in liquid nitrogen until administration.

Persons skilled in the art may make various changes without departing from the scope of the instant disclosure. Each disclosed method and method step may be performed in association with any other disclosed method or method step and in any order according to some embodiments. Where the verb “may” appear, it is intended to convey an optional and/or permissive condition, but its use is not intended to suggest any lack of operability unless otherwise indicated. Persons skilled in the art may make various changes in methods of preparing and using a composition, device, and/or system of the disclosure. Where desired, some embodiments of the disclosure may be practiced to the exclusion of other embodiments.

Also, where ranges have been provided, the disclosed endpoints may be treated as exact and/or approximations (e.g., read without or with “about”) as desired or demanded by the particular embodiment. Where the endpoints are approximate, the degree of flexibility may vary in proportion to the order of magnitude of the range. For example, on one hand, a range endpoint of about 50 in the context of a range of about to about 50 may include 50.5, but not 52.5 or 55 and, on the other hand, a range endpoint of about 50 in the context of a range of about 0.5 to about 50 may include 55, but not 60 or 75. In some embodiments, variation may simply be +/−10% of the specified value. In addition, it may be desirable, in some embodiments, to mix and match range endpoints. Also, in some embodiments, each figure disclosed (e.g., in one or more of the examples, tables, and/or drawings) may form the basis of a range (e.g., depicted value +/− about 10%, depicted value +/− about 50%, depicted value +/− about 100%) and/or a range endpoint. With respect to the former, a value of 50 depicted in an example, table, and/or drawing may form the basis of a range of, for example, about 45 to about 55, about 25 to about 100, and/or about 0 to about 100.

These equivalents and alternatives along with obvious changes and modifications are intended to be included within the scope of the present disclosure. Accordingly, the foregoing disclosure is intended to be illustrative, but not limiting, of the scope of the disclosure as illustrated by the appended claims.

The title, abstract, background, and headings are provided in compliance with regulations and/or for the convenience of the reader. They include no admissions as to the scope and content of prior art and no limitations applicable to all disclosed embodiments.

What is claimed is:

1. A method of preparing immunogenic compositions, the method comprising:
determining target genetic sequences comprising genetic sequences present in a liquid biopsy obtained from a subject;
comparing the target genetic sequences to a reference sequence comprising a wild-type genetic sequence to identify mutant genetic sequences comprising one or more non-synonymous mutations;
selecting one or more potential epitopes from the mutant genetic sequences; identifying a confirmed epitope based on an immunogenicity of the one or more potential epitopes;
producing a mutant peptide comprising the confirmed epitope; and
combining the mutant peptide with a carrier to form the immunogenic composition.

2. The method of claim 1, wherein the liquid biopsy comprises peripheral blood from the subject.

3. The method of claim 1, wherein determining target genetic sequences comprises at least one of:
enrichment of CTCs; and
enrichment of cfDNA.

4. The method of claim 3, wherein the enrichment of CTCs comprises applying positive selection based on cell size and surface protein marker expression.

5. The method of claim 3, wherein the enrichment of CTC comprises applying negative selection based on removal of white blood cells using antibody-coated magnetic beads.

6. The method of claim 3, wherein the enrichment of cfDNA comprises using at least one of:
silica-based DNA capture methods; and
carboxyl-modified-group-based DNA capture methods.

7. The method of claim 3, wherein the target genetic sequences comprise genetic sequences of at least one of the enriched cfDNA and DNA extracted from the enriched CTC.

8. The method of claim 1, wherein the target genetic sequences comprises at least one of ctDNA, cfDNA, and exosomal DNA.

9. The method of claim 1,
wherein determining target genetic sequences comprises:
enriching the liquid biopsy for CTC and cfDNA to generate an enriched liquid biopsy;
isolating ctDNA from the enriched CTC;
determining a genetic sequence for each of the ctDNA, the cfDNA, and exosomal DNA present in the liquid biopsy; and
wherein the target genetic sequences comprise the determined genetic sequences of the ctDNA, the cfDNA, and the exosomal DNA.

10. The method of claim 9, wherein determining the genetic sequence further comprises using deep sequencing comprising an average coverage of at least 10,000×.

11. The method of claim 1, wherein the wild-type genetic sequence comprises a human genome.

12. The method of claim 1, wherein selecting the one or more potential epitopes comprises removing germline mutations from the mutant genetic sequences.

13. The method of claim 12, wherein removing germline mutations comprises:
comparing the mutant genetic sequences to PBMC sequences from the subject;
identifying the germline mutations, wherein the germline mutations comprise sequences that are present in both the mutant genetic sequences and the PBMC sequences; and
removing the germline mutations from the mutant genetic sequences.

14. The method of claim 1, wherein identifying a confirmed epitope comprises:
determining the subject's HLA class type; and
determining binding affinities for the potential epitopes based on the subject's HLA class type.

15. The method of claim 14, wherein determining the subject's HLA class type comprises using one or more of:
sequence-specific primer PCR;
real time qPCR; and
next-generation sequencing.

16. The method of claim 14, wherein determining binding affinities for the mutant genetic sequences comprises:
determining a resulting peptide sequence encoded within the genetic sequences of the potential epitopes;
using a computer-based algorithm for predicting IC50 values for the resulting peptide sequence binding to HLA of the subject; and
selecting confirmed epitopes from those potential epitopes with high IC50 values.

17. The method of claim 14, wherein determining binding affinities for the mutant genetic sequences comprises:
determining a resulting peptide sequence encoded within both the genetic sequences of the potential epitopes and the wild-type genetic sequence corresponding to the potential epitopes;
using a computer-based algorithm for predicting IC50 values for the resulting peptide sequence binding to HLA of the subject; and
selecting confirmed epitopes from those potential epitopes where the genetic sequence has a high IC50 value and the corresponding wild-type peptide sequences does not have a high IC50 value.

18. The method of claim 14, further comprising measuring CTL activation by the confirmed epitope by determining the level of IFNγ secretion using one or more of:
an ELISpot assay;
a high throughput screening ELISA assay; and
intracellular cytokine flow cytometry targeting interleukin 2, tumor necrosis factor-alpha, and IFNγ.

19. The method of claim 1, wherein the carrier comprises autologous DC.

20. The method of claim 19, wherein the autologous DC comprises expanded monocytes isolated from the subject's PBMC.

21. The method of claim 1, further comprising administering the immunogenic composition to the subject.
